(19) United States
(12) Patent Application Publication
(10) Pub. No.: US 2026/0276638 A1
(43) Pub. Date: Sep. 17, 2026

(54) EXTRACTION PROCESS OF PANCRELIPASE AND EVALUATION THEREOF

(71) Applicant: Kashiv BioSciences, LLC., Piscataway, NJ (US)

(72) Inventors: Raja Reddy Kuppili, Thane (IN); Chintan Mineshkumar Vaywala, Ahmedabad (IN); Parva Yogeshchandra Purohit, Ahmedabad (IN); Mukesh Mahajan, Ahmedabad (IN); Sanjaykumar Vanrajbhat Talpara, Ahmedabad (IN)

(21) Appl. No.: 19/681,459

(22) Filed: May 19, 2026

Related U.S. Application Data
(63) Continuation of application No. 18/262,316, filed on Mar. 23, 2022.

(30) Foreign Application Priority Data
Mar. 23, 2021 (IN) 202121012561

(51) Int. Cl. G01N 33/573

(52) U.S. Cl. CPC G01N 33/573; G01N 2333/92

(57) ABSTRACT

The invention provided herein a pharmaceutical process used for an extraction of proteins from pancreatic sample and estimation of the extracted proteins. Moreover, the invention provides a use of suitable is selected from citrate-phosphate buffer and bicarbonate buffer capable to extract proteins from pancreatic sample. The invention further provides an analytical method to perform estimation of extracted proteins. This process provides an improved extraction method to quantify protein present in the pancreatic sample.

### CROSS-REFERENCE TO RELATED APPLICATIONS

This application is a continuation of U.S. patent application Ser. No. 18/262,316, filed on Jul. 20, 2023, which is a U.S. National Phase Application under 35 U.S.C. § 371 of International Patent Application No. PCT/IB2022/052651, filed on Mar. 23, 2022, which claims the benefit of Indian Patent Application number 202121012561, filed on Mar. 23, 2021, the contents of each of which are hereby incorporated by reference in their entirety.

### FIELD OF THE INVENTION

The invention relates to a pharmaceutical process used for an extraction of proteins from pancreatic sample and estimation of the extracted proteins. Moreover, the invention provides a use of suitable is selected from citrate-phosphate buffer and bicarbonate buffer capable to extract proteins from pancreatic sample. The invention further provides an analytical method to perform estimation of extracted proteins. This process provides an improved extraction method to quantify protein present in the pancreatic sample.

### BACKGROUND OF THE INVENTION

Pancreatic enzymes produced by the body are well known for the integral role they play in the digestion of the foods we eat. Pancreatic juice contains numerous enzymes, including amylase, lipase, protease, cholesterol esterase, and phospholipase, and the proenzymes trypsinogen, chymotrypsinogen, and procarboxypolypeptidase, which are converted in the small intestine to their active forms trypsin, chymotrypsin, and carboxypeptidase, respectively.

As described above, the product is a complex mixture of different proteins present in the pancreatic extracts. This poses a great challenge for organization to characterize all proteins and ensure the product quality. The extraction process requires a selection of suitable buffer in order to extract all proteins from sample. Further the characterization and estimation of pancreatic extracts is very difficult. The extraction process, analytical techniques for estimation of proteins present in pancrelipase plays very important role in developing robust consistent process and pharmaceutically acceptable biosimilar product.

Thus, the present invention provides a pharmaceutical process used for extraction of proteins from pancreatic sample and estimation of the extracted proteins. The invention provides a use of buffer capable to extract proteins from pancreatic sample in desired amount and reduced the loss of protein of interest during extraction and thereby the present method is very economic. The invention further provides an analytical method to perform estimation of extracted proteins. This process provides an improved extraction method to quantify protein present in pancreatic sample.

### SUMMARY OF THE INVENTION

In an embodiment the invention provides a process for an extraction of pancreatic protein from pancreatic protein sample comprises:
a. treating the pancreatic protein sample with suitable buffer selected from citrate-phosphate and bicarbonate buffer at suitable pH;
b. dissolving the pancreatic protein sample in the buffer;
c. collecting the extracted pancreatic protein.

In an embodiment the invention provides improved extraction process for the extraction of pancreatic protein with citrate phosphate buffer in comparison to the extraction process performed without using citrate-phosphate buffer.

In another embodiment the invention provides an improved extraction process for the extraction of pancreatic protein with bicarbonate buffer in comparison to the extraction process performed without using bicarbonate buffer.

In certain embodiment the invention provides a buffer concentration is selected from about 10 mM to about 200 mM.

In certain embodiment the pH of citrate buffer is selected from about 6.0, about 6.1, about 6.2, about 6.3, about 6.4 and about 6.5.

In certain embodiment the pH of bicarbonate buffer is selected from and about 9.5, about 9.6, about 9.7, about 9.8 and about 10.

In an embodiment, the estimation of protein performed by using BCA Kit.

In an embodiment, the process provides pancreatic protein yield at least about 80% to 100% from pancreatic protein sample.

In an embodiment the invention performs the estimation of extracted protein by using SDS-PAGE, CE-SDS, SE-HPLC and RP-HPLC method.

In an embodiment the invention provides a pharmaceutically acceptable pancreatic protein mixture comprising one or more enzymes selected from amylase, lipase and protease.

In an embodiment the quantification of pancreatic protein is performed by using method selected from SDS-PAGE, CE-SDS, SE-HPLC and RP-HPLC method.

This process provides an improved extraction method to quantify protein present in pancreatic sample by using suitable buffer selected from citrate-phosphate buffer and bicarbonate buffer.

### BRIEF DESCRIPTION OF FIGURES

FIG. 1: Depicts the reduced and non-reduced SDS PAGE analysis
1. Marker (10-180 kDa)
2. USP lipase-sodium phosphate buffer
3. USP lipase-citrate phosphate buffer
4. USP lipase-bicarbonate buffer
5. USP Protease amylase std.—sodium phosphate buffer
6. USP Protease amylase std.—citrate phosphate buffer
7. USP Protease amylase std.—bicarbonate buffer
8. Reference standard extract—sodium phosphate buffer
9. Reference standard extract—citrate phosphate buffer
10. Reference standard extract-bicarbonate buffer

FIG. 2. Representative Comparative SDS-PAGE (reduced) profile of Reference standard and samples.

FIG. 3: Separation of protein molecular weight size standard.

Based on molecular weight size standard shown in FIG. 3, apparent molecular weight of the pancreatic protein extracted with citrate phosphate buffer is identified.

FIG. 4: Overlay of Reference standard and Pancreatic samples.

FIG. 5. Non-reduced reverse phase chromatographic (RPC) profile of Reference standard enteric granule extract.

FIG. 6. Reduced reverse phase chromatographic profile of Reference standard enteric granule extract.

FIG. 7. Comparative reverse phase chromatographic (non-reduced) profile of Reference standard and samples.

FIG. 8. Comparative reverse phase chromatographic (reduced) profile of Reference standard and samples.

FIG. 9: Chromatographic overlay of inhouse and reference product analysed by SE-HPLC under non-reducing condition using 100 mM Citrate Phosphate Buffer with 10% Acetonitrile as mobile phase.

It is evident from FIG. 9 that the pancreatic protein extracted with citrate phosphate buffer shows the batch to batch consistency.

FIG. 10: Chromatographic overlay of inhouse and reference product analysed by SE-HPLC under reducing condition using 100 mM Citrate Phosphate Buffer with 10% Acetonitrile as mobile phase.

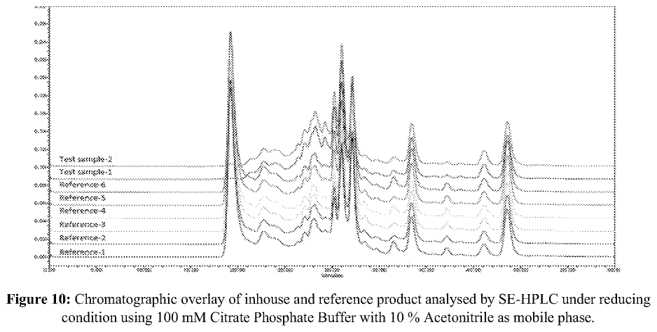

It is evident from FIG. 10 that the pancreatic protein extracted with citrate phosphate buffer shows the batch to batch consistency.

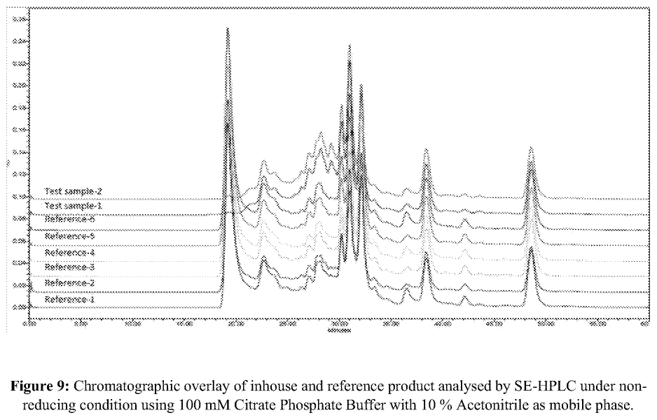

Comparative size-based qualitative profile of the constituent proteins obtained through reduced and non-reduced CE-SDS need to be represented in the form of an overlay (FIGS. 9 and 10). Similarity between protein profiles observed between Reference standard and the samples needs to be inferred.

### DETAILED DESCRIPTION OF THE EMBODIMENTS

### Definitions

Unless the context clearly requires otherwise, throughout the invention, the words “comprise”, “comprising”, and the like are to be construed in an inclusive sense as opposed to an exclusive or exhaustive sense; that is to say, in the sense of “including, but not limited to”.

The term “about” as used herein is intended to refer to ranges of approximately 10 to 20% greater than or less than the referenced value. In certain circumstances, one skill in the art will recognize that, due to the nature of the referenced value, the term about can mean more or less than a 10% to 20% deviation from that value.

The term “a suitable buffer” refers to citrate phosphate buffer, and bicarbonate buffer.

The term “Reference standard” refers pancrelipase product which are approved by regulatory agencies FDA and EMA. In certain embodiment the reference standard is selected from creon, Pancreaze, Pancrelipase, Pangestyme EC, Pangestyme C, Panocaps, Pertzye, Ultracaps, Ultresa, Viokace, Zenpep.

In an embodiment the reference standard is Creon.

The term “Pancrelipase samples” or “pancreatic sample” or “pancreatic protein sample” refers to pancreatic digestive enzymes formulated in any pharmaceutical composition. In an embodiment the pancrelipase sample is selected from granules, tablet, capsules and powder. The “Pancrelipase samples” or “pancreatic sample” or “pancreatic protein sample” comprises at one enzyme selected from lipase, protease, amylase and combination thereof. In embodiment the “Pancrelipase samples” or “pancreatic sample” or “pancreatic protein sample” obtained from crude, partially purified, substantially purified and microbially synthesize.

In an embodiment, the pancreatic protein sample comprises enzymes selected from Triacylglycerol lipase, Co-lipase, CEL lipase, Phospholipase A2, Trypsin, Chymotrypsin, Elastase, Carboxypeptidase A1, Carboxypeptidase B, Kallikrien glandular, and Alpha amylase are the prominent functionally important enzymes.

The term “substantially dissolves” refers to completely dissolving the pancreatic protein sample in buffer. In one embodiment substantially dissolves means pancreatic protein sample dissolves selected from about 99%, about 98%, about 99%, and 100% in buffer. Further dissolution of pancreatic protein in buffer is an important parameter to avoid the loss of protein of interest. Further provides suitable solution of pancreatic protein for analysis.

The term “Pancreatic protein” refers to pancrelipase sample which is substantially dissolve in citrate phosphate buffer or bicarbonate buffer thereby desired pancreatic protein is obtained through extraction which comprises at least one enzyme is selected from protease, lipase and amylase and combination thereof. In an embodiment, pancreatic protein substantially contains desired digestive enzymes in a solution suitable for performing further analysis using SDS-PAGE, CE-SDS, SE-HPLC and RP-HPLC method.

In one embodiment, the estimated yield of pancreatic protein depends on type of pancreatic protein sample used.

In one embodiment, the method provides the yield of pancreatic protein more than about 80%. For extraction for an example if granules contain 5 mg/ml, the present method at least extract more than about 80%, more than about 90% and most probably 100% of it.

In one embodiment, the method provides the yield of pancreatic protein is selected from about 80%, about 81%, about 82%, about 83%, about 84%, about 85%, about 86%, about 87%, about 88%, about 89%, about 90%, about 91%, about 92%, about 93%, about 94%, about 95%, about 99%, and about 100%.

The present invention provides the improved extraction process for extraction of proteins from pancreatic protein sample and estimation of the extracted proteins, wherein the extraction and the estimation of protein from pancreatic protein sample is performed with suitable buffer.

In one embodiment, an invention provides improved extraction process for an extraction of pancreatic protein from pancreatic protein sample comprises:
a. treating the pancreatic protein sample with suitable buffer selected from citrate-phosphate and bicarbonate buffer at suitable pH;
b. dissolving the pancreatic protein sample in the buffer;
c. collecting the extracted pancreatic protein.

In an embodiment the invention provides an improved extraction process for the extraction of pancreatic protein with citrate phosphate buffer in comparison to the extraction process performed without using citrate-phosphate buffer.

In another embodiment the invention provides an improved extraction process for the extraction of pancreatic protein with bicarbonate buffer in comparison to the extraction process performed without using bicarbonate buffer.

In certain embodiment the invention provides a buffer concentration is selected from about 10 mM to about 200 mM.

In certain embodiment the pH of citrate buffer is selected from about 6.0, about 6.1, about 6.2, about 6.3, about 6.4 and about 6.5.

In certain embodiment the pH of bicarbonate buffer is selected from and about 9.5, about 9.6, about 9.7, about 9.8 and about 10.

In an embodiment the invention performs the estimation of extracted protein by using SDS-PAGE, CE-SDS and RP-HPLC method.

In an embodiment the invention provides a pharmaceutically acceptable pancreatic protein mixture comprising one or more enzymes selected from amylase, lipase and protease.

In an embodiment, the extraction of protein is performed by using citrate phosphate buffer.

In preferable embodiment, the buffers are used for evaluating the most appropriate buffer for extraction of proteins from pancreatic protein sample comprises citrate phosphate buffer.

In an embodiment the invention provides improved extraction process for an extraction of pancreatic protein from pancreatic protein sample comprises:
a. treating the pancreatic protein sample with suitable buffer selected from citrate-phosphate at suitable pH;
b. dissolving the pancreatic protein sample in the buffer;
c. collecting the extracted pancreatic protein;
   wherein the extraction of pancreatic protein with citrate phosphate buffer is improved in comparison to the extraction process performed without using citrate-phosphate buffer.

In an embodiment, the pH of citrate phosphate buffer is maintained from and about 6.0 to about 6.5.

In an embodiment, the pH of citrate phosphate buffer is maintained from and about 6.0, about 6.1, about 6.2, about 6.3, about 6.4 and about 6.5.

In an embodiment, the pH of citrate phosphate buffer is maintained from and about 6.0, and about 6.5

In an embodiment, the concentration of citrate-phosphate buffer is selected from about 10 mM to about 200 mM.

In one embodiment, the concentration of citrate-phosphate buffer used from about 10 mM, about 15 mM, about 20 mM, about 25, about 30 mM, about 35, about 40 mM, about 45, about 50 mM, about 55, about 60 mM, about 65, about 70 mM, about 75, about 80 mM, about 85, about 90 mM, about 95, about 100 mM, about 105 mM, about 110 mM, about 115 mM, about 120 mM, about 125 mM, about 130 mM, about 135 mM, about 140 mM, about 145 mM, about 150 mM, about 155 mM, about 160 mM, about 165 mM, about 170 mM, about 175 mM, about 180 mM, about 185 mM, about 190 mM, about 195 mM, about 200 mM.

In an embodiment, the concentration of citrate-phosphate buffer is about 100 mM.

In an embodiment, the extraction of protein is performed by using bicarbonate buffer.

In an embodiment the invention provides improved extraction process for an extraction of pancreatic protein from pancreatic protein sample comprises:
a. treating the pancreatic protein sample with bicarbonate buffer at suitable pH;
b. dissolving the pancreatic protein sample in the bicarbonate buffer;
c. collecting the extracted pancreatic protein;
   wherein the extraction of pancreatic protein with bicarbonate buffer is improved in comparison to the extraction process performed without using bicarbonate buffer.

In another embodiment, the pH of bicarbonate buffer is maintained from and about 9.5 to about 10.

In another embodiment, the pH of bicarbonate buffer is maintained from and about 9.5, about 9.6, about 9.7, about 9.8 and about 10.

In another embodiment, the pH of bicarbonate buffer is maintained from and about 9.5, and about 10.

In an embodiment, the concentration of bicarbonate buffer is selected from about 10 mM to about 200 mM.

In one embodiment, the concentration of bicarbonate buffer used from about 10 mM, about 15 mM, about 20 mM, about 25, about 30 mM, about 35, about 40 mM, about 45, about 50 mM, about 55, about 60 mM, about 65, about 70 mM, about 75, about 80 mM, about 85, about 90 mM, about 95, about 100 mM, about 105 mM, about 110 mM, about 115 mM, about 120 mM, about 125 mM, about 130 mM, about 135 mM, about 140 mM, about 145 mM, about 150 mM, about 155 mM, about 160 mM, about 165 mM, about 170 mM, about 175 mM, about 180 mM, about 185 mM, about 190 mM, about 195 mM, about 200 mM.

In an embodiment, the concentration of bicarbonate buffer is 100 mM.

In an embodiment, the pancreatic sample or pancreatic protein is substantially dissolved by using suitable method known in the art e.g., shaking, stirring etc. In an embodiment one skilled person in art can use other method for dissolving the treated pancreatic sample based on the common general knowledge.

In an embodiment, the treated pancreatic sample is stirred for suitable time selected from about 20 to about 30 minutes.

In one aspect of such embodiment the pancreatic sample is stirred for suitable time selected from about 20 minutes, about 21 minutes, about 22 minutes, about 23 minutes, about 24 minutes, about 25 minutes, about 26 minutes, about 27 minutes, about 28 minutes, about 29 minutes and about 30 minutes.

In an embodiment, the treated pancreatic sample is stirred at suitable rpm selected from about 200 rpm to about 300 rpm.

In one aspect of such embodiment pancreatic sample is stirred at suitable rpm selected from 200 rpm, 210 rpm, 220 rpm, 230 rpm, 240 rpm, 250 rpm, 260 rpm, 270 rpm, 280 rpm, 290 rpm, and 300 rpm.

In an embodiment the quantification of pancreatic protein is performed by using method selected from SDS-PAGE, CE-SDS, SE-HPLC and RP-HPLC method.

In an embodiment, the estimation of protein performed by using BCA, SDS-PAGE, CE-SDS, SE-HPLC and RP-HPLC method.

In an embodiment, the invention provides a process of estimation of extracted pancreatic proteins by using BCA. In an embodiment one skilled person in art can use other kit or extraction process.

In an embodiment, the invention provides a process of estimation of extracted pancreatic proteins by using CE-SDS.

In another embodiment, the invention provides a process of estimation of extracted pancreatic proteins by using RP-HPLC.

In another embodiment, the invention provides a process of estimation of extracted pancreatic proteins by using SDS-PAGE.

In another embodiment, the invention provides an extraction of proteins by using citrate phosphate buffer and further perform the process of estimation of extracted proteins by using CE-SDS.

In another embodiment, the invention provides an extraction of proteins by using citrate phosphate buffer and further perform the process of estimation of extracted proteins by using SE-HPLC.

In another embodiment, the invention provides a process of estimation of extracted proteins by using SE-HPLC.

In another embodiment, the invention provides an extraction of proteins by using citrate phosphate buffer and further perform the process of estimation of extracted proteins by using RP-HPLC.

In another embodiment, the invention provides an extraction of proteins by using citrate phosphate buffer and further perform the process of estimation of extracted proteins by using SDS-PAGE.

In one embodiment, molecular weight of enzyme proteins is calculated based on SDS-PAGE analysis.

In another embodiment, the invention provides an extraction of proteins by using bicarbonate buffer and further perform the process of estimation of extracted proteins by using CE-SDS.

In another embodiment, the invention provides an extraction of proteins by using bicarbonate buffer and further perform the process of estimation of extracted proteins by using RP-HPLC.

In another embodiment, the invention provides an extraction of proteins by using bicarbonate buffer and further perform the process of estimation of extracted proteins by using SE-HPLC. In another embodiment, the invention provides an extraction of proteins by using bicarbonate buffer and process of estimation of extracted proteins by using SDS-PAGE. In an embodiment, in protein extraction method for short term storage before analysis place the samples at about 2° C. to about 8° C. and for long term usage these sample can be stored in a −80° C. refrigerator.

In an embodiment the invention provides a pharmaceutically acceptable pancreatic protein mixture comprising one or more enzymes selected from amylase, lipase and protease.

### EXAMPLES

### Example 1: Process for Extraction and Estimation of Proteins from Pancrelipase Granules

Materials and reagents used for the process of the extraction and estimation are:

Water, Citric Acid, Na₂HPO₄, Micro Filter (0.2 μm), Microfuge tubes, Weighing Boat, Syringe, BCA Kit, 10×PBS Buffer, 15 mL centrifuge tubes, and 50 mL centrifuge tubes.

Evaluation of buffers: Several buffers were used for evaluating the most appropriate buffer for extraction of proteins from Pancrelipase granules given in Table 1 below:

| Buffer | pH | Conditions | Observations |
|---|---|---|---|
| Water | Milli Q | | insoluble |
| Chilled water | Milli Q | | insoluble |
| Citrate buffer | 3.0 | | insoluble |
| Citrate phosphate | 6.0 | Pancrelipase | soluble |
| buffer (Mc Ilvain's | | granules subjected | |
| buffer) | | to 100 rpm in the | |
| Citrate phosphate | 6.5 | respective buffers | soluble |
| buffer (Mc Ilvain's | | for 30 min | |
| buffer) | | | |
| Sodium Phosphate | 6.8 | | soluble with slight |
| buffer | | | turbidity |
| Sodium Phosphate | 7.0 | | soluble with slight |
| buffer | | | turbidity |
| Sodium Phosphate | 7.5 | | soluble with slight |
| buffer | | | turbidity |
| Bicarbonate buffer | 9.5 | | soluble |
| Bicarbonate buffer | 10.0 | | soluble |
| Crushed granules | 3.0, 6.0, 9.0 | Crushed granules | Except for bicarbonate |
| | | solubilized in water, | buffer all the buffers |
| | | phosphate, citrate, | led to extremely turbid |
| | | and bicarbonate buffer | solution |

Amongst the several buffers used for evaluating the most appropriate buffer for extraction of proteins from pancrelipase granules, it was observed that the granules dissolved better in citrate-phosphate buffer, and bicarbonate buffer. These were further observed for release of proteins and assessment through SDS-PAGE and CE-SDS. Also analysed with RP-HPLC method wherein, SDS-PAGE and CE-SDS were used for separation of protein species based on their molecular sizes whereas RP-HPLC was used for separation of proteins based on their hydrophobicity. The released proteins were estimated by uv spectrophotometer at 280 nm.

Preparation of Extraction Buffer, e.g., 100 Mm Citrate Phosphate Buffer: For the preparation of 100 mM of Citrate Phosphate Buffer, Na2HPO4 of 7.098 g was weighed and transferred into a glass beaker containing 300 mL of purified water. This was stirred well on a magnetic stirrer and allowed to dissolve properly. 100 mM citric acid solution was used to adjust the pH to 6.20. The volume was made up to 500 mL with purified water and the solution filtered with 0.2μ filter using vacuum pump. 100 mM Citric Acid Solution was prepared by dissolving 3.84 g of citric acid in 150 mL of purified water. This was mixed well, and volume made up to 200 mL with purified water.

Process of Protein Extraction: For the protein extraction method, we accurately weighed 125.0 mg of pancrelipase granules each of reference and samples, respectively and the granules transferred to a 100 mL volumetric flask. 25 mL of Citrate phosphate buffer was added to the volumetric flask, and stoppered. This was clamped on a wrist shaker equipment and stirred at 300 RPM for at least 30 minutes. The solution was checked for dissolution of granules have dissolved properly and the solution appears slight cream pale colour. The citrate phosphate buffer solution (1 ml) was syringe filtered with a 0.2 μm microfilter unit as equilibration step before proceeding for filtration of sample solution. The reference/sample solution was taken in this syringe and the filtrate was collected extracted reference/sample solution. This protein concentration of the filtered sample or extract was estimated by using BCA kit method. For short term storage before analysis place the samples at 2-8° C. and for long term usage these sample can be stored in a −80° C. refrigerator. Avoid multiple freeze thaw cycles by preparing aliquots after extraction and freezing them.

### Process of Protein Estimation:

a. Preparation of BCA working reagent: For the preparation of BCA reagent, 50 mL of reagent A (BCA solution) was taken and added 1 mL of reagent B [copper (II) sulphate pentahydrate 4% solution] and mixed well in a 50 mL centrifuge tube.

b. Preparation of 1×PBS buffer: buffer 2 mL of (10×PBS) was taken then 18 mL of purified water was added to it, mixed it well.

c. Preparation of BSA standards for the standard plot given in Table 2 below:

| | | |
|---|---|---|
| Solution Concentration | BSA Standard | |
| (mg/mL) | (1 mg/mL) | 1X PBS |

| | | | | |
|---|---|---|---|---|
| 0.2 | 40 | μL | 100 | μL |
| 0.4 | 80 | μL | 120 | μL |
| 0.6 | 120 | μL | 80 | μL |
| 0.8 | 160 | μL | 40 | μL |

| | | | |
|---|---|---|---|
| 1.0 | 200 | μL | -NA- |

d. Preparation of reaction blank solution: 100 μL of 1×PBS was taken and 2 mL of BCA working reagent was added to it and mixed it well.

e. Preparation of reference/samples for protein estimation: 50 μL of protein samples were taken and added 150 μL of diluent (1×PBS) to it and mixed it well. This dilution is in a ratio of 1:3. The volumes can be varied keeping the ratio intact. Taken 100 μL of above solution and added 2 mL of BCA working reagent to it and mixed well. Incubated all preparations which is BSA linearity standard solutions, Blank solution, reference, and samples, at room temperature for 2.5-3 h. The incubation can be extended up to 4 h but not less than 2.5 h. After the incubation time, measured the absorbance measurement of all the above solutions at 562 nm wavelength in a UV spectrophotometer.

f. Determination of protein concentration: Sample dilution factor (for 1:3 ratio the dilution factor would be 4) was applied to obtain the final reportable protein concentration in mg/mL. The standard curve between absorbance at 562 nm Vs. Concentration (mg/mL) was determined for linearity (R2), slope (m) and intercept (c) value, for the BSA standards. Based on the OD at 562 nm values obtained for the reference/samples, back calculated concentration was taken the values of the standard curve into consideration as follows. The OD at 562 nm for a sample was found to be 0.441, and the concentration was calculated based on the formula:

$$\begin{matrix}y = mx + c \\ x = ( y - c ) / m \\ = ( 0.441 - 0.0373 ) / 0.7695 \\ = 0.5246 \\ x = 0.5246 \times Dilution Factor \\ = 0.5246 * 4 \\ = 2.1 mg / Ml\end{matrix}$$

For Slope (m): 0.7695
For Intercept (c): 0.0373
Dilution factor: 4

Result: Among all the buffered used the citrate-phosphate buffer and the bicarbonate buffer are best for the extrations.

It is evident from FIG. 1 that both reducing and non-reducing SDS-PAGE that the intensity of protein bands is comparatively higher in pancreatic mixture of USP lipase, protease and amylase standard extracted in citrate-phosphate buffer.

Intensity of bands in a pancreatic mixture of reference standard extracted with citrate-phosphate buffer and bicarbonate buffer was higher in comparison to sodium-phosphate buffer indicating that both extraction buffers can be used.

### Example 2: Estimation of Protein by Using SDS-PAGE Method Comprises Following Step

Materials and Reagent used for the SDS-PAGE method are: Purified Water, 10×Tris/Glycine buffer, Pre-cast gel (4-20%), Sample Reducing agent (10×), NuPAGE, LDS sample buffer, Protein ladder-4 color, 10-180 kDa, Citric Acid, Na2HPO4, Staining solution, Methanol, and Glacial acetic acid.

a. Preparation of Diluent buffer e.g., 100 Mm Citrate Phosphate Buffer preparation process is mentioned above.

b. For the Preparation of running buffer e.g., 1× Tris Glycine buffer—For the preparation of running buffer added 100 mL of 10×Tris Glycine buffer and made up the volume to 1000 mL with purified water then mixed it well and transferred it in a bottle.

c. Destaining solution: 100 mL of purified water was taken and added 80 mL of methanol, mixed it well. Added 20 mL of Glacial acetic acid to this solution and mixed well in a glass bottle.

d. Preparation of Pancrelipase test sample: For the preparation of pancrelipase test sample we referred method for protein extraction and estimation we mentioned above. Protein concentration value for pancrelipase extract is determined by BCA kit method. Considered this value for further dilution of non-reducing and reducing samples.

e. Preparation of Reducing Sample: Diluted the sample to 1.0 mg/mL with citrate phosphate buffer for a total sample volume of 100 μL. 18 μL of this sample was taken and added 6 μL of sample reducing agent and mixed it well, incubated this sample at 90° C. for 4 min. Allowed it to cool down to room temperature and give a short spin to the sample tubes. This sample is loaded based in the well based on the sample concentration to achieve final on-gel protein load of 10-15 μg. For samples of 1 mg/mL added about 10 μL and for samples below 1 mg/mL add volume such as to achieve final on-gel protein load of 10-15 μg.

### Details of SDS-PAGE Given in Table 3 Below:

| | |
|---|---|
| Gel | 4-20% precast gel |
| Running buffer | 1X Tris Glycine SDS buffer |
| Voltage | 140 V |
| Run time | About 1 h or till loading dye front reaches |
| | the line marked on the precast gel cassette |

After completion of the run the pre-cast gel cassette is broken on the arrows indicated and rinsed in purified water carefully. Transferred the gel in a box containing staining solution. After sufficient staining of the gel (about 20 min), staining solution is removed. The gel is then rinsed with purified water shortly before transferring it into destaining solution. Allowed to destain till the background coloration is removed and the bands appear clearly. Transferred the gel in purified water and keep it overnight, if required, before taking gel picture.

The extracted protein was evaluated for the citrate phosphate, sodium phosphate buffer, and bicarbonate buffer by SDS-PAGE (4-20%). This was also compared to the USP standards in the same buffers to have a size-based comparison. It was observed to give a better protein yield in citrate phosphate buffer, in the size range corresponding to the expected theoretical protein sizes (FIG. 1).

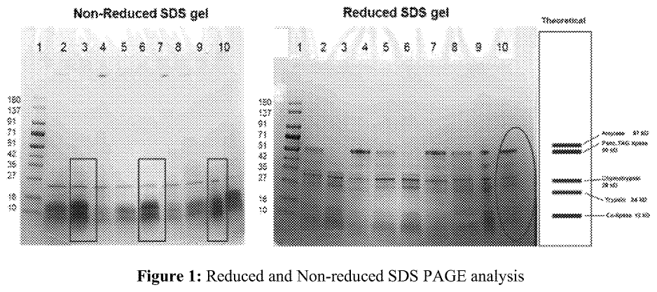

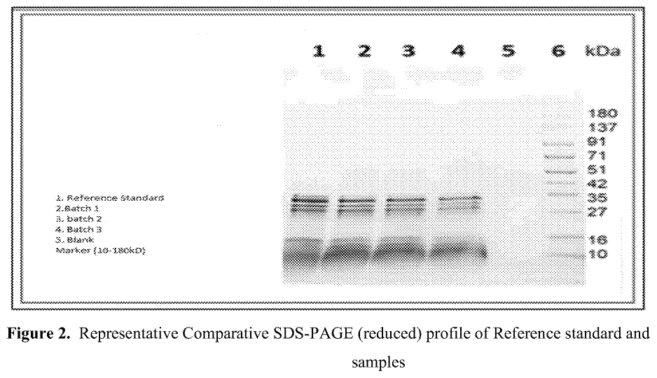

It is evident from FIG. 2 that the pancreatic protein extracted with citrate phosphate buffer shows the batch to batch consistency.

### Example 3: Estimation of Protein by Using CE-SDS Method

CE-SDS was utilized for understanding the size-based protein species and was further used for comparison of the reference and in-house protein product. CE-SDS assist in demonstrating similarity and differences based on size variants.

Materials and reagents used for CE-SDS method are: IgG Purity/Heterogeniety Kit, SDS-MW Gel Buffer Multipack, MW Size Standard, 10 kDa standard, Pre-Assembled Capillary Cartridge, Vials, Caps, Micro Vials, Iodoacetamide, Milli Q Water, and Betamercapto ethanol.

### The Procedure for Estimation of Protein by Using CE-SDS Method:

Preparation of the Alkylation reagent (250 mM IAM solution): Weighed 46 mg of iodoacetamide (IAM). Transferred IAM to a centrifuge tube. Added 1.0 mL of milli Q water to the centrifuge tube. Cap the vial tightly, mixed thoroughly until dissolved using vortex, and then stored in the dark place. The IAM solution is stable for approximately 24 hours at room temperature. This solution should be prepared fresh.

1. Preparation of SDS MW size standard: Pipptte 10 μL of Size standard into a centrifuge vial. Added 85 μL of sample buffer into the centrifuge vial. Added 5 μL of 2-mercaptoethanol. Cap the vial tightly, seal with Parafilm, and mixed thoroughly. Heated mixture in a water bath at 100° C. for three minutes. Placed the vial in a room-temperature to cool for five minutes before injection. Transferred 90 μL of the sample into a micro vial.

2. Preparation of blank: Pipptte 45 μL of buffer into a centrifuge vial. Added 55 μL of sample buffer into the centrifuge vial. Added 5 μL of 250 mM IAM solution. Cap the vial tightly, seal with Parafilm, and mixed thoroughly. Heated mixture in a water bath at 70° C. for three minutes. Placed the vial in a room-temperature to cool for five minutes before injection. Transferred 90 μL of the sample into a micro vial.

3. Preparation of sample: Diluted the sample to get the any concentration in the range of 0.5 mg/mL to 1.5 mg/mL using milli Q water. Samples should preferably be diluted to 1.0 mg/mL or to any fixed concentration in above range based on objective of study. Final salt concentration should preferably be less than 50 mM. Samples above 80 mM should be diluted to final salt concentration less than 80 mM and concentration not less than 0.5 mg/mL.

Pipptte 45 μL of buffer into a centrifuge vial. Added 55 μL of sample buffer into the centrifuge vial. Added 5 μL of 250 mM IAM solution. Cap the vial tightly, seal with parafilm, and mixed thoroughly. Heated mixture in a water bath at 70° C. for three minutes. Placed the vial in a room-temperature to cool for five minutes before injection. Transferred 90 μL of the sample into a micro vial.

### 4. Preparation of Buffer Trays (Inlet and Outlet):

i. Buffer Inlet Tray: Prepared the buffer inlet tray as per the schematic shown in the Table 4 below and loaded on the left side of the instrument.

TABLE 4
Inlet buffer tray configuration

| No. | A | B | C | D | E | F |
|---|---|---|---|---|---|---|
| 6 | H₂O | H₂O | | | | |
| | (Cycle 24- | (Cycle 24- | | | | |
| | 35) | 35) | | | | |
| 5 | H₂O | H₂O | | | | |
| | (Cycle 12- | (Cycle 12- | | | | |
| | 23) | 23) | | | | |
| 4 | H₂O | H₂O | | | | |
| | (Cycle 1- | (Cycle 1- | | | | |
| | 11) | 11) | | | | |
| 3 | H₂O | Gel-R | Gel-S | NaOH | HCl | H₂O |
| | (Cycle 24- | (Cycle 24- | (Cycle 24- | (Cycle 24- | (Cycle 24- | (Cycle 24- |
| | 35) | 35) | 35) | 35) | 35) | 35) |
| 2 | H₂O | Gel-R | Gel-S | NaOH | HCl | H₂O |
| | (Cycle 12- | (Cycle 12- | (Cycle 12- | (Cycle 12- | (Cycle 12- | (Cycle 12- |
| | 23) | 23) | 23) | 23) | 23) | 23) |
| 1 | H₂O | Gel-R | Gel-S | NaOH | HCl | H₂O |
| | (Cycle 1- | (Cycle 1- | (Cycle 1- | (Cycle 1- | (Cycle 1- | (Cycle 1- |
| | 11) | 11) | 11) | 11) | 11) | 11) |

Al to A6-Water, use in dip step to clean capillary tip, 1.5 mL
B4 to B6-Water, use in dip step to clean capillary tip, 1.5 mL
B1 to B3-Gel-R (SDS-MW gel buffer), use to rinse/fill capillary prior to each cycle, 1.2 mL
C1 to C3-Gel-S (SDS-MW gel buffer), use for separation, 1.1 mL
Dl to D3-0.1N NaOH, use to precondition capillary, 1.5 mL
E1 to E3-0.1N HCl, use to precondition capillary, 1.5 mL
Fl to F3-Water, use to precondition capillary, 1.5 mL

ii. Buffer Outlet Tray: Prepared the buffer outlet tray as per the schematic shown in the Table 5 below and loaded on the right side of the instrument.

TABLE 5
Outlet buffer tray configuration

| No. | A | B | C | D | E | F |
|---|---|---|---|---|---|---|
| 6 | H₂O | H₂O | | | | |
| | (Cycle 24- | (Cycle 24- | | | | |
| | 35) | 35) | | | | |
| 5 | H₂O | H₂O | | | | |
| | (Cycle 12- | (Cycle 12- | | | | |
| | 23) | 23) | | | | |
| 4 | H₂O | H₂O | | | | |
| | (Cycle 1- | (Cycle 1- | | | | |
| | 11) | 11) | | | | |
| 3 | H₂O | Waste | Gel-S | Waste | Waste | Waste |
| | (Cycle 24- | (Cycle 24- | (Cycle 24- | (Cycle 24- | (Cycle 24- | (Cycle 24- |
| | 35) | 35) | 35) | 35) | 35) | 35) |
| 2 | H₂O | Waste | Gel-S | Waste | Waste | Waste |
| | (Cycle 12- | (Cycle 12- | (Cycle 12- | (Cycle 12- | (Cycle 12- | (Cycle 12- |
| | 23) | 23) | 23) | 23) | 23) | 23) |
| 1 | H₂O | Waste | Gel-S | Waste | Waste | Waste |
| | (Cycle 1- | (Cycle 1- | (Cycle 1- | (Cycle 1- | (Cycle 1- | (Cycle 1- |
| | 11) | 11) | 11) | 11) | 11) | 11) |

Al to A6-Water, used in dip step to clean capillary tip, 1.5 mL
B4 to B6-Water, used in dip step to clean capillary tip, 1.5 mL
B1 to B3-Water, waste vial for SDS-MW gel buffer rinse, 0.8 mL
Cl to C3-Gel-S (SDS-MW gel buffer), used for separation, 1.1 mL
Dl to D3-Water, waste vial for 0.1N NaOH rinse, 0.8 mL
El to E3-Water, waste vial for 0.1N HCl rinse, 0.8 mL
Fl to F3-Water, waste vial for Water rinse, 0.8 mL

Based on number of samples, filled up the buffer tray (Inlet and Outlet). The system automatically replenishes all reagents through an increment of the buffer array tray after every eleven cycles or samples.

### Injection Sequence: Table 6 Below Shows Injection Sequence:

| Sr. No. | Sample name | Runtime |
|---|---|---|
| | Conditioning | 10 |
| 1 | Blank | 30 |
| 2 | MW Standard | 30 |
| 3 | Test Sample-1 | 30 |
| | Conditioning | 10 |
| n-1 | Test Sample-n (n ≤ 12) | 30 |
| n | MW Standard (after every 10 | 30 |
| | samples and end of the sequence) | |

Bracketing with MW standard was not required up to 5 samples. Samples include MW standard, blanks and test samples.

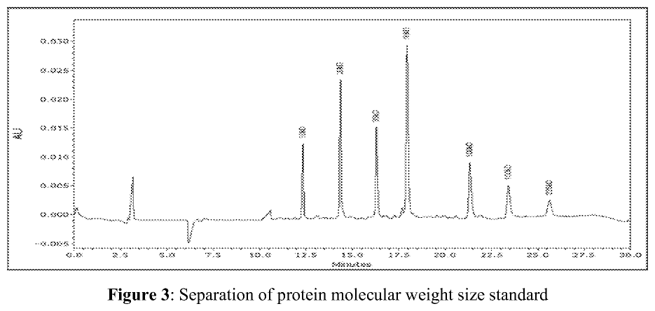

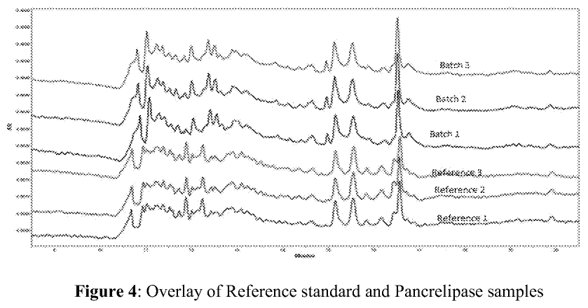

Separation of protein molecular weight size standard as shown in FIGS. 3 and 4.

Result: It is evident from FIG. 4 that using CE-SDS method the pancreatic protein extracted with citrate phosphate buffer shows the batch to batch consistency.

### Example 4: Estimation of Protein by Using RP-HPLC

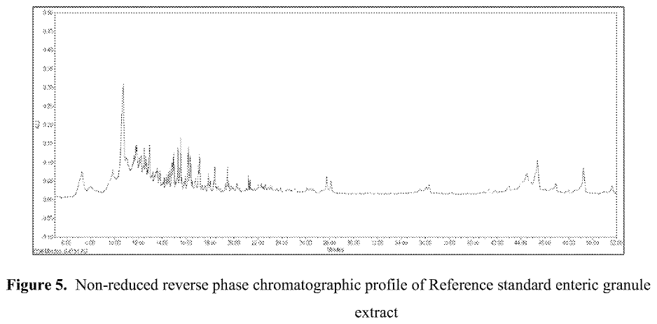

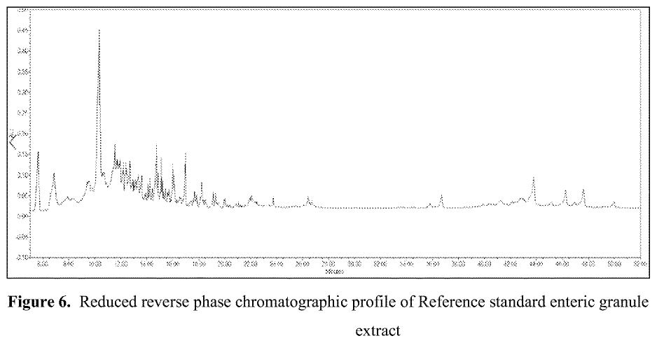

RP-HPLC is used to evaluate the qualitative attribute, indicating protein identity between reference and samples. Therefore, the chromatographic profile obtained should be reported, without any integration (Refer FIGS. 5 and 6).

Materials and reagent details: Water, Acetonitrile, TFA, DTT, Citric Acid, Na2HPO4, and HCl. The Procedure for estimation of protein by using RP-HPLC:

1. Preparation of diluent buffer e.g., 100 Mm Citrate Phosphate Buffer mentioned above.

2. Mobile Phase Preparation: i) Mobile Phase A: 0.1% TFA in Water: Added 1.0 mL of TFA in 1000 mL of purified water. Mixed it well and sonicated for 5 minutes. ii) Mobile Phase B: 0.1% TFA in Acetonitrile: Added 1.0 mL of TFA in 1000 mL of Acetonitrile. Mixed it well and sonicated for 5 minutes.

3. Preparation of 1 M DTT: Weighed 154.3 mg of DTT and dissolved it in 1.0 mL of purified water. Mixed well. Prepared fresh every time.

4. Preparation of Pancrelipase test sample: For the extraction of proteins referred method for protein extraction and estimation for pancrelipase given above. Protein concentration value for pancrelipase extract is determined by BCA kit method. Considered this value for further dilution of non-reducing and reducing samples.

i. Non-Reducing Sample: Made the final concentration to 1.0 mg/mL with the diluent buffer (100 mM Citrate Phosphate buffer).

ii. Reducing Sample: Diluted the sample to 1.0 mg/mL with purified water with a final DTT concentration of 10 mM in the sample. For this added 4 μL of 1 M DTT to 396 μL of 1 mg/mL sample. Incubated it for 30 minutes at 37° C. in water bath or dry bath. This sample is to be injected based on initial concentration to achieve final on-column load amount of 20 μg.

### 5. Method Details for RP-HPLC:

Referred both the Tables 7 and Table 8 below for the details of chromatographic parameters.

TABLE 7
Details of Chromatographic parameters

| Column description | Acquity BEH UPLC C18 column | |
|---|---|---|
| | 2.1 × 150 mm, 1.7 μm, 300 Å | |
| Column temperature | 70° | C. |
| Sample manager temperature | 7° | C. |
| Flow rate | 0.3 | mL/min |
| Wavelength | 214 | nm |

TABLE 8
Chromatography gradient details

| Time | Mobile phase A | Mobile phase B | | |
|---|---|---|---|---|
| (min) | (%) | (%) | Flow (mL/min) | Curve |
| 0.0 | 100 | 0 | 0.3 | 6 |
| 3.0 | 100 | 0 | | |
| 18.0 | 70 | 30 | | |
| 20.0 | 63 | 37 | | |
| 50.0 | 55 | 45 | | |
| 65.0 | 25 | 75 | | |
| 70.0 | 0 | 100 | | |
| 75.0 | 0 | 100 | | |
| 76.0 | 100 | 0 | | |
| 80.0 | 100 | 0 | | |

Injection Sequence: 1) Injected one or two Blanks at the beginning of a sequence or till a stable baseline is achieved. 2) Injected reference standard solution before the injection of the first sample and at the end of a sequence.

### Required Injected Blank and Injection Sequence are Given Below in Table 9.

| Sr. No. | Solution | No. of injection |
|---|---|---|
| 1 | Blank (Mobile phase A) | 2 |
| 2 | Citrate phosphate buffer | 1 |
| 3 | Reference standard solution | 1 |
| 5 | Blank | 1 |
| 6 | Sample 1 | 1 |
| 7 | Sample 2 | 1 |
| 8 | Sample 3 | 1 |
| 9 | Blank | 1 |
| 10 | Reference standard solution Bracketing | 1 |

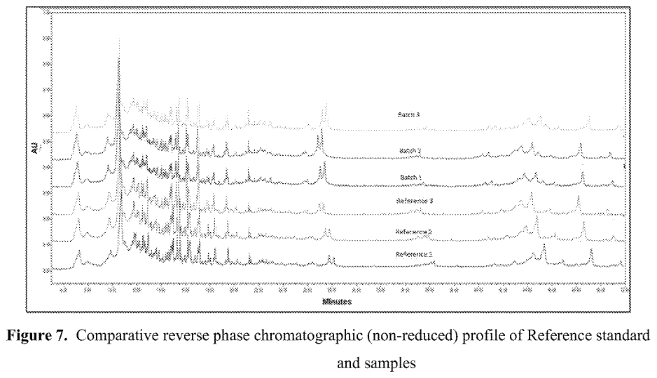

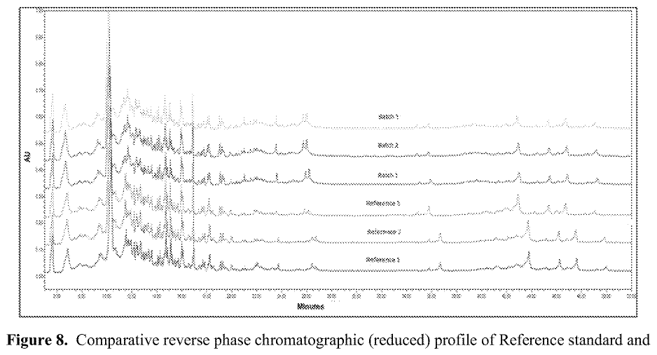

Results are shown in FIGS. 5 and 6. Data reporting and interpretation shown FIGS. 7 and 8.

FIG. 5 indicates that the various proteins present in the pancreatic protein extracted with citrate phosphate buffer are identified under non-reducing condition by RPC.

FIG. 6 indicates that the various proteins present in the pancreatic protein extracted with citrate phosphate buffer are identified under reducing condition by RPC.

It is evident from FIG. 7 that the pancreatic protein extracted with citrate phosphate buffer shows the batch to batch consistency.

It is evident from FIG. 8 that the pancreatic protein extracted with citrate phosphate buffer shows the batch to batch consistency.

Comparative hydrophobicity based qualitative profile of the constituent proteins obtained through reduced and non-reduced reverse phase chromatography need to be represented in the form of an overlay (FIGS. 7 and 8). Similarity between protein profiles observed between Reference standard and the samples needs to be inferred.

1. A process for an extraction of a pancreatic protein from a pancreatic protein sample, the process comprising:
a. dissolving the pancreatic protein sample in the buffer;
b. collecting the extracted pancreatic protein,
wherein the buffer is selected from citrate-phosphate buffer and bicarbonate buffer;
wherein the pancreatic protein sample comprises a pharmaceutical composition comprising:
one or more enzymes selected from lipase, protease, and amylase; and
one or more enzymes selected from Triacylglycerol lipase, Co-lipase, CEL lipase, Phospholipase A2, Trypsin, Chymotrypsin, Elastase, Carboxypeptidase A1, Carboxypeptidase B, Kallikrien glandular, and Alpha amylase.

2. The process according to claim 1, wherein the extraction of the pancreatic protein is improved in comparison to an extraction process performed without using a buffer selected from citrate-phosphate buffer and bicarbonate buffer.

3. The process according to claim 1, wherein:
the citrate-phosphate buffer concentration is selected from about 10 mM to about 200 mM; or
the bicarbonate buffer concentration is selected from about 10 mM to about 200 mM.

4. The process according to claim 3, wherein:
the citrate-phosphate buffer concentration is selected from about 100 mM; or
the bicarbonate buffer concentration is selected from about 100 mM.

5. The process according to claim 1, wherein the citrate phosphate buffer has a pH from about 6.0 to about 6.5.

6. The process according to claim 1, wherein the bicarbonate buffer has a pH from about 9.5 to about 10.

7. The process according to claim 1, wherein dissolving the pancreatic protein sample in the buffer is performed by stirring or shaking.

8. The process according to claim 7, wherein stirring is performed at 200 rpm to 300 rpm.

9. The process according to claim 8, wherein stirring is performed for about 20 minutes to about 30 minutes.

10. The process according to claim 1, wherein the extracted pancreatic protein is further subjected to estimation of protein performed by using BCA, SDS-PAGE, CE-SDS, SE-HPLC, or RP-HPLC.

11. The process according to claim 1, wherein the extracted pancreatic protein is further subjected to quantification of protein performed by using SDS-PAGE, CE-SDS, SE-HPLC, or RP-HPLC.

12. The process according to claim 1, wherein the process provides more than about 80% yield of pancreatic protein.

13. The process according to claim 1, wherein the process provides more than about 90% yield of pancreatic protein.

14. The process according to claim 1, wherein the process provides more than about 98% yield of pancreatic protein.

15. The process according to claim 1, wherein the extracted pancreatic protein comprises a protein selected from amylase, lipase and protease.

16. The process according to claim 1, wherein the process provides a pharmaceutically acceptable pancreatic protein mixture comprising one or more enzymes selected from amylase, lipase and protease.

17. The process according to claim 10, wherein the extracted pancreatic protein is further subjected to estimation of protein based on molecular weight by using CE-SDS.

18. The process according to claim 10, wherein the extracted pancreatic protein is further subjected to estimation of purity profile by using RP-HPLC.

19. The process according to claim 10, wherein the extracted pancreatic protein is further subjected to estimation of protein based on molecular weight by using SDS-PAGE.

20. The process according to claim 10, wherein the extracted pancreatic protein is further subjected to estimation of purity profile by using SE-HPLC.
